(19) United States
(12) Patent Application Publication
(10) Pub. No.: US 2015/0065830 A1
(43) Pub. Date: Mar. 5, 2015

(54) SYSTEM AND METHOD FOR OPERATING A SENSOR FOR DETERMINING BLOOD CHARACTERISTICS OF A SUBJECT

(71) Applicant: General Electric Company, Schenectady, NY (US)

(72) Inventors: Jason Harris Karp, Niskayuna, NY (US); Bahman Ebrahimi Kashef, Newburyport, MA (US); SM Shajedul Hasan, Rexford, NY (US)

(73) Assignee: General Electric Company, Schenectady, NY (US)

(21) Appl. No.: 14/014,562

(22) Filed: Aug. 30, 2013

(51) Int. Cl. A61B 5/1455; A61B 5/024

(52) U.S. Cl. CPC A61B 5/14552; A61B 5/02427
USPC 600/331; 600/479

(57) ABSTRACT

In accordance with one aspect of the present technique, a method is disclosed. The method includes receiving continuous photoplethysmographic (PPG) data of a subject from a sensor and calculating a continuous blood characteristic (BC) based on the continuous PPG data. The method also includes calculating a first quality metric of the continuous PPG data based on a sequence of the continuous BC. The method further determines whether the first quality metric satisfies a stability criterion and sending a first notification to the sensor in response to determining that the first quality metric satisfies the stability criterion. The first notification instructs the sensor to collect compressed PPG data of the subject.

This invention was made with partial Government support under contract number W81XWH1110833 awarded by U.S. Department of the Army. The Government has certain rights in the invention.

### BACKGROUND

The subject matter disclosed herein generally relates to operating a sensor for determining blood characteristics of a subject. More specifically, the subject matter relates to systems and methods for switching the operation of a pulse oximeter sensor for determining blood characteristics of a subject based on the quality of the subject's plethysmographic data.

Doctors, primary physicians, and the like, often use sensors (e.g., pulse oximeter sensor) to monitor blood characteristics, for example, oxygen saturation level, heart rate, and the like, of their patients. Existing pulse oximeter sensors have numerous problems. For example, the pulse oximeter sensors that continuously operate during a cardiac cycle of a patient consume significant amounts of power to pulse the light emitting diodes of the pulse oximeter sensor. In another example, the pulse oximeter sensors that operate during only the systolic phase of a cardiac cycle face challenges in synchronizing the pulsing of LEDs with the heart rate of the subject.

Thus, there is a need for an enhanced system and method for operating a sensor for determining blood characteristics of a subject.

### BRIEF DESCRIPTION

In accordance with one aspect of the present technique, a method includes receiving continuous photoplethysmographic (PPG) data of a subject from a sensor and calculating a continuous blood characteristic (BC) based on the continuous PPG data. The method also includes calculating a first quality metric of the continuous PPG data based on a sequence of the continuous BC. The method further includes determining whether the first quality metric satisfies a stability criterion and sending a first notification to the sensor in response to determining that the first quality metric satisfies the stability criterion. The first notification instructs the sensor to collect compressed PPG data of the subject.

In accordance with one aspect of the present systems, a system includes a calculation module configured to receive continuous PPG data of a subject from a sensor, calculate a continuous BC based on the continuous PPG data, and calculate a first quality metric of the continuous PPG data based on a sequence of the continuous BC. The system also includes a determination module configured to determine whether the first quality metric satisfies a stability criterion and send a first notification to the sensor in response to determining that the first quality metric satisfies the stability criterion. The first notification instructs the sensor to collect compressed PPG data of the subject.

In accordance with one aspect of the present technique, a computer program product encoding instructions is disclosed. The instructions when executed by a processor, causes the processor to receive continuous PPG data of a subject from a sensor and calculate a continuous BC based on the continuous PPG data. The instructions further cause the processor to calculate a first quality metric of the continuous PPG data based on a sequence of the continuous BC. The instructions further cause the processor to determine whether the first quality metric satisfies a stability criterion and send a first notification to the sensor in response to determining that the first quality metric satisfies the stability criterion. The first notification instructs the sensor to collect compressed PPG data of the subject.

### DRAWINGS

These and other features, aspects, and advantages of the present disclosure will become better understood when the following detailed description is read with reference to the accompanying drawings in which like characters represent like parts throughout the drawings, wherein:

FIG. 1 is a block diagram illustrating a system for determining blood characteristics of a subject according to one embodiment;

FIG. 2 is a graphical representation of the operation of a sensor according to one embodiment;

FIG. 3 is a graphical representation of the operation of a sensor according to another embodiment;

FIG. 4 is a graphical representation of a calibration function according to one embodiment;

FIG. 5 is a flow diagram of a method for operating a sensor for determining blood characteristics according to one embodiment; and

FIG. 6 is a graphical representation for operating a sensor for determining blood characteristics according to one embodiment.

### DETAILED DESCRIPTION

In the following specification and the claims, reference will be made to a number of terms, which shall be defined to have the following meanings.

The singular forms “a”, “an”, and “the” include plural references unless the context clearly dictates otherwise.

As used herein, the term “non-transitory computer-readable media” is intended to be representative of any tangible computer-based device implemented in any method or technology for short-term and long-term storage of information, such as computer-readable instructions, data structures, program modules and sub-modules, or other data in any device. Therefore, the methods described herein may be encoded as executable instructions embodied in a tangible, non-transitory, computer readable medium, including, without limitation, a storage device and/or a memory device. Such instructions, when executed by a processor, cause the processor to perform at least a portion of the methods described herein. Moreover, as used herein, the term “non-transitory computer-readable media” includes all tangible, computer-readable media, including, without limitation, non-transitory computer storage devices, including, without limitation, volatile and nonvolatile media, and removable and non-removable media such as a firmware, physical and virtual storage, CD-ROMs, DVDs, and any other digital source such as a network or the Internet, as well as yet to be developed digital means, with the sole exception being a transitory, propagating signal.

As used herein, the terms “software” and “firmware” are interchangeable, and may include any computer program stored in memory for execution by devices that include, without limitation, mobile devices, clusters, personal computers, workstations, clients, and servers.

As used herein, the term “computer” and related terms, e.g., “computing device”, are not limited to integrated circuits referred to in the art as a computer, but broadly refers to at least one microcontroller, microcomputer, programmable logic controller (PLC), application specific integrated circuit, and other programmable circuits, and these terms are used interchangeably herein.

Approximating language, as used herein throughout the description and claims, may be applied to modify any quantitative representation that could permissibly vary without resulting in a change in the basic function to which it is related. Accordingly, a value modified by a term or terms, such as “about” and “substantially”, are not limited to the precise value specified. In at least some instances, the approximating language may correspond to the precision of an instrument for measuring the value. Here and throughout the specification and claims, range limitations may be combined and/or inter-changed, such ranges are identified and include all the sub-ranges contained therein unless context or language indicates otherwise.

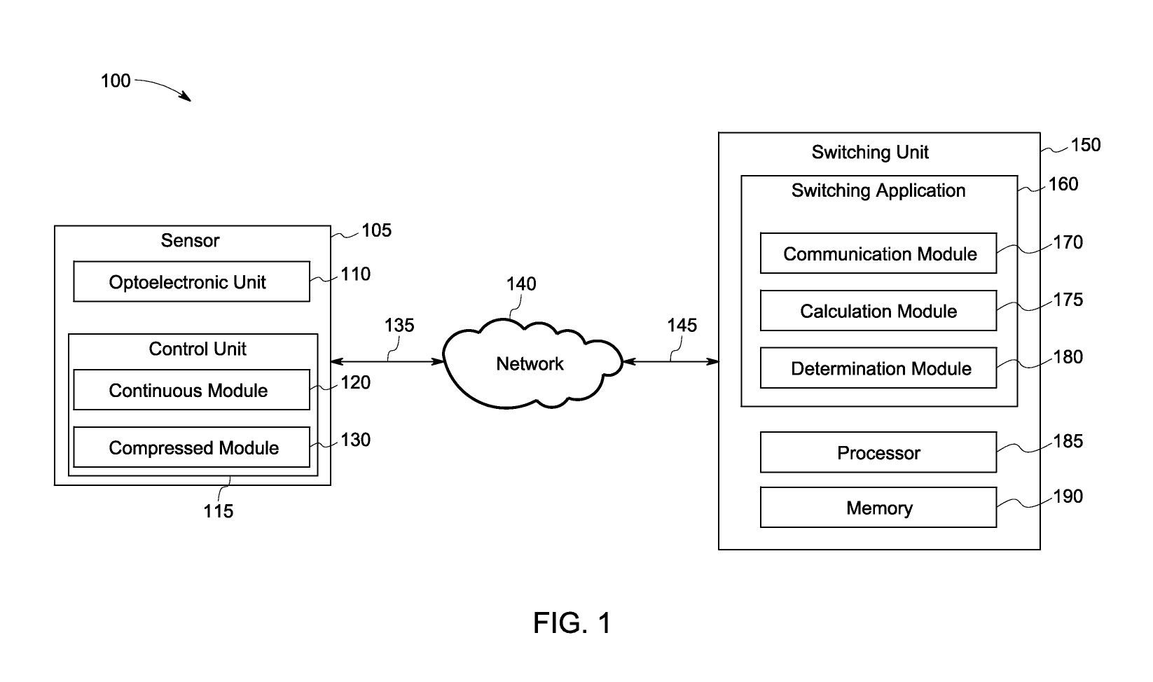

A system and method for operating a sensor for determining blood characteristics (e.g., oxygen saturation, heart rate, and the like) of a subject (e.g., a patient in a hospital and the like) is described herein. FIG. 1 illustrates a block diagram of a system 100 configured to determine blood characteristics of a subject according to one embodiment. The system 100 includes a sensor 105 and a switching unit 150 that are communicatively coupled via a network 140.

The network 140 may be a wired or wireless communication type, and may have any number of configurations such as a star configuration, token ring configuration, or other known configurations. Furthermore, the network 140 may include a local area network (LAN), a wide area network (WAN) (e.g., the Internet), and/or any other interconnected data path across which multiple devices may communicate. In one embodiment, the network 140 may be a peer-to-peer network. The network 140 may also be coupled to or include portions of a telecommunication network for transmitting data in a variety of different communication protocols. In another embodiment, the network 140 includes Bluetooth communication networks or a cellular communications network for transmitting and receiving data such as via a short messaging service (SMS), a multimedia messaging service (MMS), a hypertext transfer protocol (HTTP), a direct data connection, WAP, email, and the like. While only one network 140 is shown coupled to the sensor 105 and the switching unit 150, multiple networks 140 may be coupled to the entities.

The sensor 105 may be any type of device for collecting PPG data of a subject. Typically, the PPG data indicates the change in volume within a body part of the subject due to fluctuations in the amount of blood, air, and the like, within the body part. In the illustrated embodiment, the sensor 105 is a pulse oximeter sensor including an optoelectronic unit 110 and a control unit 115 configured to collect photoplethysmographic (PPG) data of the subject. The sensor 105 is further configured to send the collected PPG data of the subject to the switching unit 150 via the network 140. The sensor 105 is coupled to the network 140 via a signal line 135. The signal line 135 is provided for illustrative purposes and represents the sensor 105 communicating by wires or wirelessly over the network 140.

The optoelectronic unit 110 includes a plurality of light emitting elements (not shown), for example, light emitting diodes (LED) for emitting light through a body part (e.g., finger, ear lobe, and the like) of a subject. In one embodiment, the optoelectronic unit 110 includes two LEDs for emitting light at wavelengths of 660 nm (red) and 940 nm (infrared). Although, the optoelectronic unit 110 is described herein as including two LEDs emitting red light and infrared light, in other embodiments, the optoelectronic unit 110 may include LEDs emitting light at any wavelength. The optoelectronic unit 110 further includes at least one photo detector (not shown) for receiving the light emitted by the LEDs after passing through a body part of the subject and converting them into electrical signals, i.e., PPG data.

The control unit 115 includes a continuous module 120 and a compressed module 130 configured to control the operation of the optoelectronic unit 110 (i.e., switching on and switching off of the plurality of LEDs) to collect the PPG data during one or more cardiac cycles of a subject. A cardiac cycle refers to a sequence of events related to the flow of blood that occurs from the beginning of one heartbeat to the beginning of the next heartbeat of a subject. The cardiac cycle includes a systolic phase and a subsequent diastolic phase. During the systolic phase, the heart ventricles contract and pump blood into the arteries of the subject. During the diastolic phase, the ventricles of the heart relax and get filled with blood. In one embodiment, the control unit 115 may include a memory (not shown) and a processor (not shown) for storing and executing the codes and routines of the continuous module 120 and the compressed module 130. Although, the control unit 115 is described above according to one embodiment as a part of the sensor 105, in other embodiments, the control unit 105 may be included in the switching unit 150.

The continuous module 120 includes codes and routines to operate the optoelectronic unit 110 to collect PPG data throughout one or more cardiac cycles, i.e., during the systolic and the diastolic phases. The continuous module 120 periodically switches on and switches off the plurality of LEDs and receives corresponding PPG waveforms (i.e., PPG data) recorded by the photo detector.

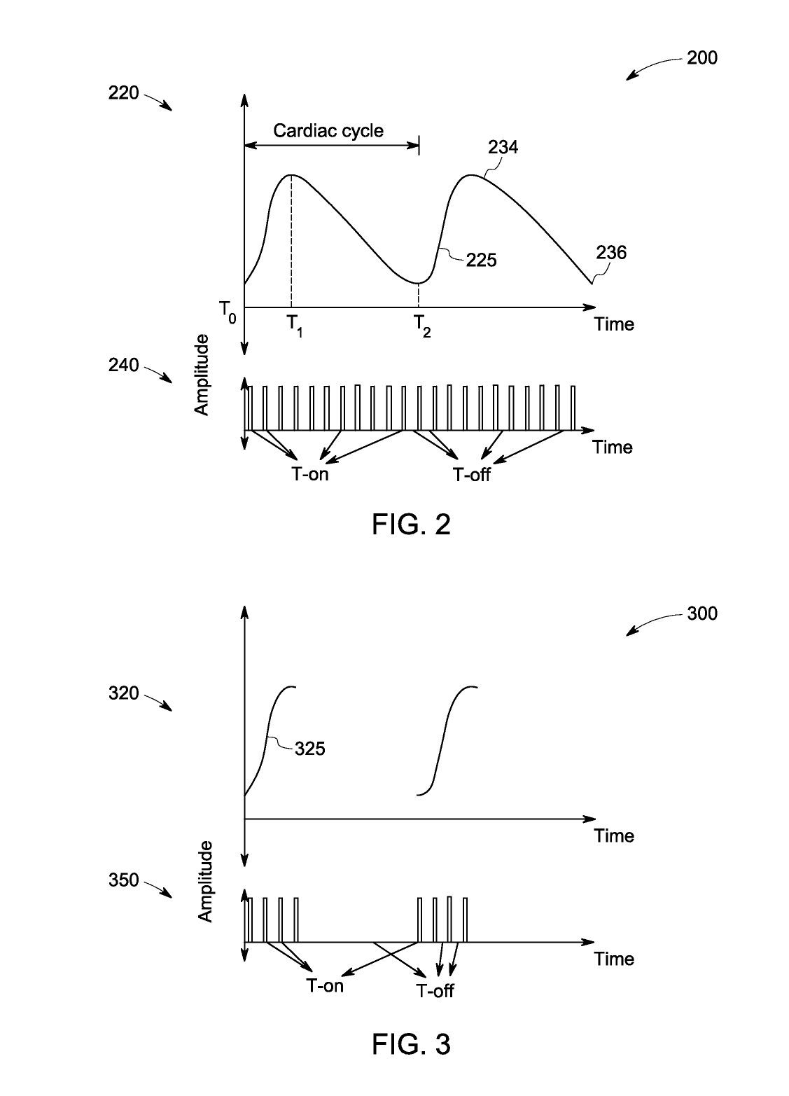

Referring now to FIG. 2, a graphical representation 200 of the operation of the optoelectronic unit is illustrated according to one embodiment. The graph 220 illustrates a PPG waveform 225 (i.e., PPG data) received by the continuous module by operating an LED emitting, for example, red light over two successive cardiac cycles. Each cardiac cycle includes a systolic phase (represented by the portion of the PPG waveform 225 between time instants Tₒ and T₁) and a diastolic phase (represented by the portion of the PPG waveform 225 between time instants T₁ and T₂).

The graph 240 illustrates the time instants at which the continuous module switches on (T-on) and switches off (T-off) the LED to record the PPG waveform 225. In the illustrated embodiment, the continuous module switches on the LED during both systolic and diastolic phases of each cardiac cycle for recording the PPG waveform 225. Although, FIG. 2 illustrates only one PPG waveform 225 recorded by operating an LED emitting red light, the continuous module receives another PPG waveform by similarly operating another LED emitting, for example, infrared light. For the purpose of clarity and convenience, the two PPG waveforms received by the continuous module are collectively referred to herein as “continuous PPG data”.

Referring back to FIG. 1, the compressed module 130 includes codes and routines configured to operate the optoelectronic unit 110 to collect PPG data during the systolic phase of a cardiac cycle.

FIG. 3 illustrates a graphical representation 300 of the operation of the optoelectronic unit according to another embodiment. The graph 320 illustrates a PPG waveform 325 received by the compressed module by operating an LED emitting, for example, red light over two successive cardiac cycles. The graph 350 illustrates the time instants at which the compressed module switches on (T-on) and switches off (T-off) for recording the PPG waveform 325. In the illustrated embodiment, the compressed module switches on the LED only during the systolic phase of each cardiac cycle. Although, FIG. 3 illustrates only one PPG waveform 325 recorded by operating an LED emitting red light, the compressed module receives another PPG waveform by similarly operating another LED emitting, for example, infrared light. For the purpose of clarity and convenience, the two PPG waveforms received by the compressed module are collectively referred to herein as “compressed PPG data”.

Referring again to FIG. 1, the switching unit 150 may be any device configured to switch the operation of the sensor 105 based on the quality of a subject's PPG data for determining blood characteristics of the subject. The switching unit 150 receives the subject's PPG data from the sensor 105 via the network 140. The switching unit 150 is communicatively coupled to the network 140 via a signal line 145. The signal line 145 is provided for illustrative purposes and represents the switching unit 150 communicating by wires or wirelessly over the network 140. In the illustrated embodiment, the switching unit 150 includes a switching application 160, a processor 185, and a memory 190. The switching application 160 includes a communication module 170, a calculation module 175, and a determination module 180. The plurality of modules of the switching application 160, the processor 185, and the memory 190 are coupled to a bus (not shown) for communication with each other.

The processor 185 may include at least one arithmetic logic unit, microprocessor, general purpose controller or other processor arrays to perform computations, and/or retrieve data stored on the memory 190. In another embodiment, the processor 185 is a multiple core processor. The processor 185 processes data signals and may include various computing architectures including a complex instruction set computer (CISC) architecture, a reduced instruction set computer (RISC) architecture, or an architecture implementing a combination of instruction sets. The processing capability of the processor 185 in one embodiment may be limited to supporting the retrieval of data and transmission of data. The processing capability of the processor 185 in another embodiment may also perform more complex tasks, including various types of feature extraction, modulating, encoding, multiplexing, and the like. In other embodiments, other type of processors, operating systems, and physical configurations are also envisioned.

The memory 190 may be a non-transitory storage medium. For example, the memory 190 may be a dynamic random access memory (DRAM) device, a static random access memory (SRAM) device, flash memory or other memory devices. In one embodiment, the memory 190 also includes a non-volatile memory or similar permanent storage device, and media such as a hard disk drive, a floppy disk drive, a compact disc read only memory (CD-ROM) device, a digital versatile disc read only memory (DVD-ROM) device, a digital versatile disc random access memory (DVD-RAM) device, a digital versatile disc rewritable (DVD-RW) device, a flash memory device, or other non-volatile storage devices.

The memory 190 stores data that is required for the switching application 160 to perform associated functions. In one embodiment, the memory 190 stores the modules (e.g., communication module 170, calculation module 175, and the like) of the switching application 160. In another embodiment, the memory 190 stores stability criteria (e.g., standard deviation threshold value, environmental threshold value, time threshold value, and the like) that are defined, for example, by an administrator of the switching unit 150. The stability criteria are described below in further detail with reference to the determination module 180.

The communication module 170 includes codes and routines configured to handle communications between the sensor 105 and other modules of the switching application 160. In one embodiment, the communication module 170 includes a set of instructions executable by the processor 185 to provide the functionality for handling communications between the sensor 105 and other modules of the switching application 160. In another embodiment, the communication module 170 is stored in the memory 190 and is accessible and executable by the processor 185. In either embodiment, the communication module 170 is adapted for communication and cooperation with the processor 185 and other modules of the switching application 160.

In one embodiment, the communication module 170 receives PPG data from the control unit 115 of the sensor 105 via the network 140. For example, the communication module 170 receives the PPG data in real-time corresponding to each cardiac cycle of the subject. The received PPG data includes either continuous PPG data recorded by the continuous module 120 or compressed PPG data recorded by the compressed module 130. In such an embodiment, the communication module 170 sends the PPG data to the calculation module 175. In another embodiment, the communication module 170 receives a notification instruction for switching the operation of the sensor 105 from the determination module 180. In such an embodiment, the communication module 170 sends the notification to the control unit 115 of the sensor 105 via the network 140.

The calculation module 175 includes codes and routines configured to calculate one or more blood characteristics (BCs) and calculating one or more quality metrics of the PPG data. In one embodiment, the calculation module 175 includes a set of instructions executable by the processor 185 to provide the functionality for calculating one or more BCs and one or more quality metrics of the PPG data. In another embodiment, the calculation module 175 is stored in the memory 190 and accessible and executable by the processor 185. In either embodiment, the calculation module 175 is adapted for communication and cooperation with the processor 185 and other modules of the switching application 160.

The calculation module 175 receives PPG data from the communication module 170 and calculates one or more BCs (e.g., percentage modulation, oxygen saturation, heart rate, and the like) from the received PPG data. In one embodiment, the calculation module 175 receives continuous PPG data that includes a continuous PPG waveform recorded by operating an LED emitting red light and another continuous PPG waveform recorded by operating an LED emitting infrared light. For the purpose of clarity and convenience, a BC calculated from the continuous PPG data is referred to herein as a continuous BC.

In one embodiment, the calculation module 175 calculates a percentage modulation (i.e., a perfusion index) of each continuous PPG waveform for each cardiac cycle as a continuous BC. The calculation module 175 calculates a percentage modulation based on the equation:

$$\% mod = \frac{{AC}_{max} - {AC}_{min}}{DC}$$

### Where:

% mod is the percentage modulation of the continuous PPG waveform;

ACₘₐₓ is the maximum value of the continuous PPG waveform;

ACₘᵢₙ is the minimum value of the continuous PPG waveform; and

DC is the offset level of the continuous PPG waveform.

Referring again to FIG. 2, the calculation module calculates the % mod_{R} of the continuous PPG waveform 225 (i.e., received by operating the LED emitting red light) for the second cardiac cycle using the ACₘₐₓ value 234 and the ACₘᵢₙ value 236. Similarly, the calculation module 175 calculates the % mod_{IR} for each cardiac cycle of the continuous PPG waveform received by operating the LED emitting infrared light.

In another embodiment, the calculation module 175 calculates a ratio between the % mod_{R} and the % mod_{IR} for each cardiac cycle as a continuous BC. In a further embodiment, the calculation module 175 calculates an oxygen saturation (Spo₂ value) of a subject based on the ratio between % mod_{R} and the % mod_{IR} using a calibration function.

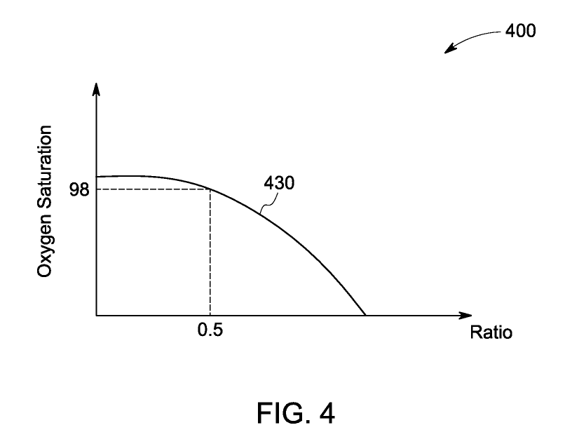

FIG. 4 illustrates a graph 400 depicting the calibration function 430 according to one embodiment. In the illustrated embodiment, if the ratio between the % mod_{R} and the % mod_{IR} is 0.5, the calculation module calculates the oxygen saturation as 98 based on the calibration function 430.

Referring again to FIG. 1, in one embodiment, the calculation module 175 calculates a heart rate of the subject based on the continuous PPG data as a continuous BC. In such an embodiment, the calculation module 175 determines the time duration between each cardiac cycle of the continuous PPG data for determining the heart rate.

In one embodiment, the calculation module 175 receives compressed PPG data from the communication module 170. The calculation module 175 calculates one or more BCs from the compressed PPG data similar to the aforementioned calculation of the one or more continuous BCs. For the purpose of clarity and convenience, a BC calculated from the compressed PPG data is referred to herein as a “compressed BC”. For example, the calculation module 175 calculates the % mod_{R} as a compressed BC based on the compressed PPG waveform 325 (See FIG. 3).

The calculation module 175 further calculates one or more quality metrics of the received PPG data. In one embodiment, the calculation module 175 calculates a quality metric of the continuous PPG data based on a sequence of continuous BCs corresponding to a sequence of cardiac cycles of the subject. In such an embodiment, the calculation module 175 determines an average BC (e.g., arithmetic mean, a weighted arithmetic mean, geometric mean, median, mode, etc.) of the sequence of continuous BCs. The calculation module 175 then calculates a standard deviation of each continuous BC in the sequence using the average BC as the quality metric. For example, the calculation module 175 determines an average Spo₂ value for a sequence of three Spo₂ values. The calculation module 175 then calculates a standard deviation of each of the three Spo₂ values as the quality metric of the received continuous PPG data. Although, the quality metric based on the sequence of continuous BCs is described above using Spo₂ values according to one example, in other examples, the calculation module 175 can calculate the quality metric using % mod values, ratio between % mod_{R} and the % mod_{IR}, heart rate, and the like.

In another embodiment, the calculation module 175 calculates a quality metric of the continuous PPG data by determining a presence of environmental signals in the received continuous PPG data. The environmental signals include, for example, noise signals caused due to electrical circuitry of the optoelectronic unit 110, motion artifacts caused due to the movement of the subject, and the like. In such an embodiment, the calculation module 175 transforms the two continuous PPG waveforms (i.e., continuous PPG data) into a Fourier domain to determine the presence of the environmental signals. The calculation module 175 calculates the amplitude of the environmental signal as a quality metric of the received continuous PPG data.

In one embodiment, the calculation module 175 receives compressed PPG data from the communication module 170. In such an embodiment, the calculation module 175 calculates one or more quality metrics for the compressed PPG data similar to the aforementioned calculation of one or more quality metrics for the continuous PPG data. For example, the calculation module 175 determines an average heart rate value (i.e., compressed BC) for a sequence of five heart rate values. The calculation module 175 then calculates a standard deviation of each of the five heart rate values as the quality metric of the received compressed PPG data. In another example, the calculation module 175 determines a presence of an environmental signal in the compressed PPG data and calculates the amplitude of the environmental signal as a quality metric of the compressed PPG data.

The calculation module 175 sends the one or more quality metrics of the PPG data to the determination module 180. In one embodiment, the calculation module 175 generates graphical data for displaying the one or more BCs to, for example, a doctor. In such an embodiment, the calculation module 175 sends the graphical data to a display device (not shown) coupled to either the switching unit 150 or the sensor 105.

The determination module 180 includes codes and routines configured to determine whether one or more quality metrics of the PPG data satisfies a stability criteria and switch the operation of the sensor 105. In one embodiment, the determination module 180 includes a set of instructions executable by the processor 185 to provide the functionality for determining whether one or more quality metrics of the received PPG data satisfies a stability criteria and for switching the operation of the sensor 105. In another embodiment, the determination module 180 is stored in the memory 190 and accessible and executable by the processor 185. In either embodiment, the determination module 180 is adapted for communication and cooperation with the processor 185 and other modules of the switching application 160.

In one embodiment, the determination module 180 receives one or more quality metrics of continuous PPG data. The determination module 180 determines whether the one or more quality metrics of the continuous PPG data satisfy one or more stability criteria. The stability criteria (e.g., standard deviation threshold value, environmental threshold value, time threshold value, and the like) are defined by, for example, an administrator of the switching unit 160. The determination module 180 sends a first notification to the sensor 105 in response to determining that the one or more quality metrics satisfy the stability criteria. In such an embodiment, the first notification instructs the sensor 105 to operate the compressed module of the optoelectronic unit 115 and collect compressed PPG data of the subject.

For example, the determination module 180 receives the standard deviations of the heart rate of a subject over three cardiac cycles as 1, 0, and 1. In such an example, the determination module 180 determines that the standard deviation of each heart rate is within the standard deviation threshold value of 3. The determination module 180 then sends the first notification to the sensor 105 to stop the collection of continuous PPG data and start the collection of compressed PPG data. In another example, the determination module 180 receives the amplitude of an environmental signal present in the received continuous PPG data. If the determination module 180 determines that the amplitude of the environmental signal is lesser than the environmental threshold value, the determination module 180 sends the first notification to the sensor 105.

In another embodiment, the determination module 180 receives one or more quality metrics of compressed PPG data. The determination module 180 determines whether the one or more quality metrics of the compressed PPG data satisfy one or more stability criteria. The determination module 180 sends a second notification to the sensor 105 in response to determining that the one or more quality metrics fail to satisfy the stability criteria. In such an embodiment, the second notification instructs the sensor 105 to operate the continuous module 120 of the optoelectronic unit 115 and collect continuous PPG data of the subject.

For example, the determination module 180 receives the standard deviations of the heart rate of a subject over four cardiac cycles as 1, 0, 1 and 5. In such an example, the determination module 180 determines that the standard deviation of the heart rate during the fourth cardiac cycle exceeds the standard deviation threshold value of 3. The determination module 180 then sends the second notification to the sensor 105 to stop the collection of compressed PPG data and start the collection of continuous PPG data. In another example, the determination module 180 receives the amplitude of an environmental signal present in the received compressed PPG data. If the determination module 180 determines that the amplitude of the environmental signal exceeds the environmental threshold value, the determination module 180 sends the second notification to the sensor 105.

In one embodiment, the determination module 180 sends the second notification to the sensor 105 based on an elapsed time. The elapsed time indicates the time duration for which the sensor 105 has been collecting compressed PPG data of the subject. The determination module 180 calculates the elapsed time in response to sending the first notification to the sensor 105. In such an embodiment, the determination module 180 determines whether the elapsed time is within a time threshold value. The determination module 180 sends the second notification to the sensor 105 in response to determining that the elapsed time has exceeded the time duration value. In another embodiment, the determination module 180 receives a user input, for example, from a doctor, for collecting continuous PPG of a subject. In such an embodiment, the determination module 180 sends the second notification to the sensor 105.

In yet another embodiment, the determination module 180 determines whether the switching unit 150 receives the PPG data continuously from the sensor 105. For example, the determination module 180 determines whether the communication module 170 receives PPG data in real-time corresponding to every cardiac cycle of the subject. The determination module 180 sends the first notification in response to determining that that the communication module 170 fails to receive the PPG data in real-time. The communication module 170 may fail to receive the PPG data continuously due to, for example, a failure in the functioning of the network 140, signal lines, 135, 145 and the like. The first notification instructs the sensor 105 to collect compressed PPG data of the subject. Such an embodiment is advantageous as temporarily storing compressed PPG data in the memory (not shown) of the sensor 105 requires lesser storage space than storing continuous PPG data.

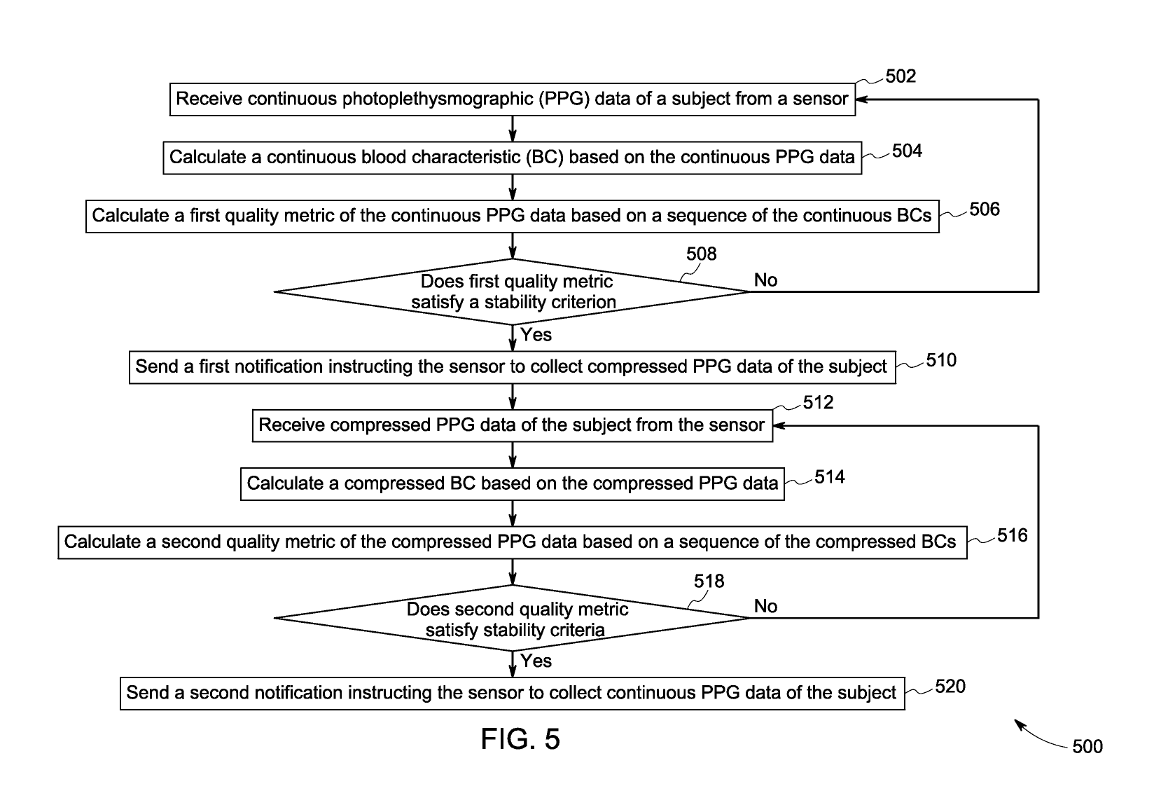

FIG. 5 illustrates a flow diagram 500 of a method for operating a sensor for determining BCs according to one embodiment. The communication module receives continuous PPG data of a subject from a sensor 502. The calculation module calculates a continuous BC based on the continuous PPG data 504. For example, the calculation module calculates the Spo₂ value from the continuous PPG data as the continuous BC. The calculation module further calculates a first quality metric of the continuous PPG data based on a sequence of the continuous BCs 506. In the above example, calculation module calculates the standard deviation for a sequence of five continuous Spo₂ values as the first quality metric. The determination module determines whether the first quality metric satisfies a stability criterion 508. In the above example, the determination module determines whether the standard deviation of each continuous Spo2 value is within a standard deviation threshold value.

If the first quality metric fails to satisfy the stability criterion, the communication module continues to receive continuous PPG data of the subject from the sensor 502. If the first quality metric satisfies the stability criterion, the determination module sends a first notification instructing the sensor to collect compressed PPG data of the subject 510. The communication module then receives compressed PPG data of the subject from the sensor 512. The calculation module calculates a compressed BC based on the compressed PPG data 514. For example, the calculation module calculates the Spo₂ value from the compressed PPG data as the compressed BC. The calculation module further calculates a second quality metric of the compressed PPG data based on a sequence of the compressed BCs 516. In the above example, calculation module calculates the standard deviation for a sequence of five compressed Spo₂ values as the second quality metric.

The determination module then determines whether the second quality metric satisfies a stability criterion 518. In the above example, the determination module determines whether the standard deviation of each compressed Spo₂ value is within the standard deviation threshold value. If the second quality metric satisfies the stability criterion, the communication module continues to receive compressed PPG data of the subject from the sensor 512. If the second quality metric fails to satisfy the stability criterion, the determination module sends a second notification instructing the sensor to collect continuous PPG data of the subject 520.

Although the method 500 is described as switching the operation of the sensor based on single type of quality metric (i.e., standard deviation of continuous Spo₂ and compressed Spo₂) according to one embodiment, in other embodiments, the sensor operation may be switched based on a combination of multiple quality metrics. For example, the calculation module calculates the Spo₂ value and the heart rate value from the received continuous PPG data as continuous BCs. The calculation module then calculates the standard deviation for a sequence of Spo₂ and heart rate values as the first quality metric. In such an example, the determination module sends the first notification if the standard deviation of each Spo₂ value and each heart rate value is within the standard deviation threshold value. Although, in this example, determination module compares the standard deviation of the Spo₂ values and the heart rate values with the same standard deviation threshold value, in other examples, the determination module may compare them with different standard deviation threshold values.

In another example, the calculation module calculates the Spo₂ value from the received compressed PPG data as a compressed BC. The calculation module calculates the standard deviation for a sequence of Spo₂ values and the amplitude of an environmental signal in the compressed PPG data as the second quality metric. In such an example, the determination module sends the second notification if either the standard deviations of the Spo₂ values exceed the standard deviation threshold value or if the amplitude of the environmental signal exceeds the environmental threshold value.

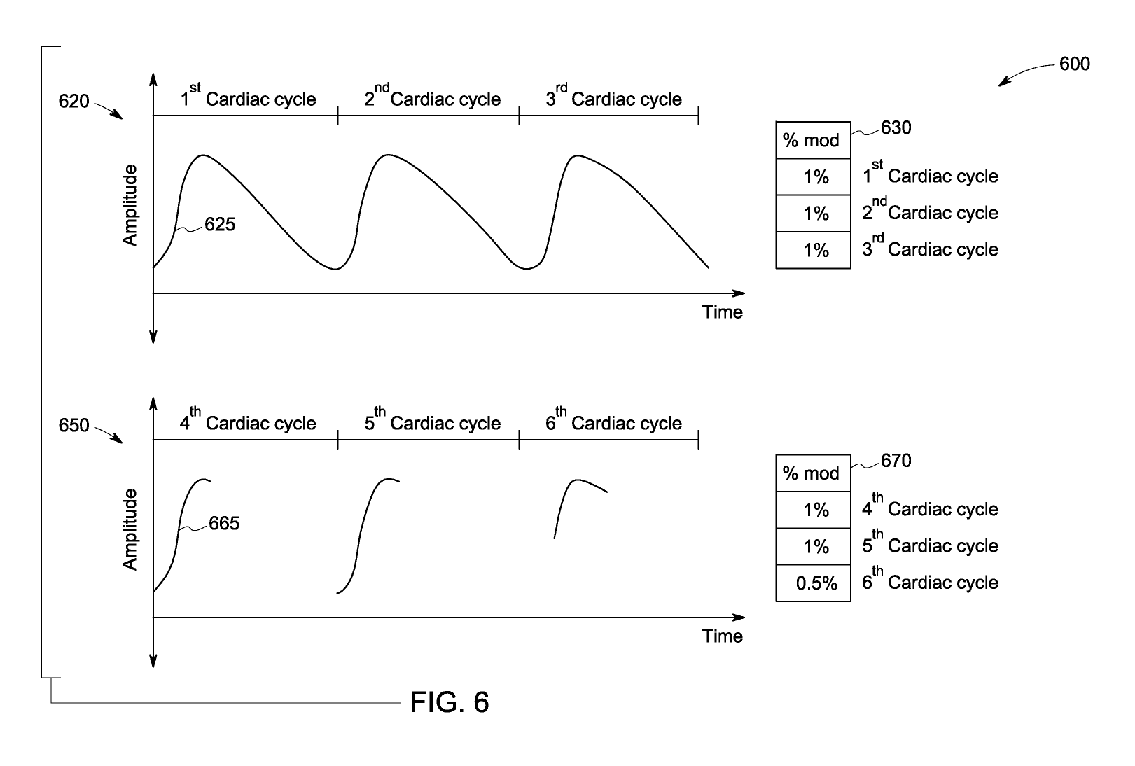

Referring now to FIG. 6, a graphical representation 600 for operating a sensor for determining BCs is illustrated according to one embodiment. The graph 620 illustrates a continuous PPG waveform 625 received by the switching unit over three successive cardiac cycles (i.e., cardiac cycles 1-3) of a subject. The continuous PPG waveform 625 is recorded by operating the LED emitting, for example, infrared light using the continuous module. The calculation module determines the % mod_{IR} of the continuous PPG waveform 625 as the continuous BC. The table 630 illustrates the sequence of % mod_{IR} values of the continuous PPG waveform 625. The calculation module further calculates the standard deviation for each of the % mod_{IR} values shown in the table 630, as the first quality metric. The determination module determines that the first quality metric is within the standard deviation threshold value and hence satisfies the stability criterion. The determination module then sends a first notification instructing the sensor to collect compressed PPG data of the subject.

The graph 650 illustrates the compressed PPG waveform 665 received by the switching unit over three successive cardiac cycles of the subject (i.e., cardiac cycles 4-6). The compressed PPG waveform 665 is recorded by operating the LED emitting infrared light using the compressed module. The control unit of the sensor switches the operation of the LED from the continuous module to the compressed module in response to receiving the first notification from the switching unit. The calculation module then calculates % mod_{IR} of the compressed PPG waveform 665 as the compressed BC. The table 670 illustrates the sequence of % mod_{IR} values of the compressed PPG waveform 665. The calculation module further calculates the standard deviation for each of the % mod_{IR} values shown in the table 670, as the second quality metric. The determination module determines that the standard deviation of the % mod_{IR} value during the sixth cardiac cycle exceeds the standard deviation threshold value and hence fails to satisfy the stability criterion. The determination module then sends a second notification instructing the sensor to collect continuous PPG data of the subject.

The above described method for switching the operation of a sensor based on the quality of the PPG data is advantageous compared to existing methods for determining BCs due to lesser power consumption by the LEDs and higher reliability and accuracy of the determined BCs.

It is to be understood that not necessarily all such objects or advantages described above may be achieved in accordance with any particular embodiment. Thus, for example, those skilled in the art will recognize that the systems and techniques described herein may be embodied or carried out in a manner that achieves or optimizes one advantage or group of advantages as taught herein without necessarily achieving other objects or advantages as may be taught or suggested herein.

While the subject matter has been described in detail in connection with only a limited number of embodiments, it should be readily understood that the inventions are not limited to such disclosed embodiments. Rather, the subject matter can be modified to incorporate any number of variations, alterations, substitutions or equivalent arrangements not heretofore described, but which are commensurate with the spirit and scope of the inventions. Additionally, while various embodiments of the subject matter have been described, it is to be understood that aspects of the inventions may include only some of the described embodiments. Accordingly, the inventions are not to be seen as limited by the foregoing description, but are only limited by the scope of the appended claims.

What is claimed as new and desired to be protected by Letters Patent of the United States is:

1. A method comprising:
receiving continuous photoplethysmographic (PPG) data of a subject from a sensor;
calculating a continuous blood characteristic (BC) based on the continuous PPG data;
calculating a first quality metric of the continuous PPG data based on a sequence of the continuous BC;
determining whether the first quality metric satisfies a stability criterion; and
sending a first notification to the sensor in response to determining that the first quality metric satisfies the stability criterion, wherein the first notification instructs the sensor to collect compressed PPG data of the subject.

2. The method of claim 1, further comprising:
receiving compressed PPG data of the subject from the sensor in response to sending the first notification;
calculating a compressed BC based on the compressed PPG data;
calculating a second quality metric of the compressed PPG data based on a sequence of the compressed BC;
determining whether the second quality metric satisfies the stability criterion; and
sending a second notification to the sensor in response to determining that the second quality metric fails to satisfy the stability criterion, wherein the second notification instructs the sensor to collect continuous PPG data of the subject.

3. The method of claim 2, wherein the continuous BC and the compressed BC includes at least one of an oxygen saturation, a percentage modulation, and a heart rate.

4. The method of claim 2, wherein calculating the second quality metric further comprises calculating a standard deviation of the sequence of the compressed BC.

5. The method of claim 2, wherein calculating the second quality metric further comprises calculating an environmental signal from the compressed PPG data.

6. The method of claim 2, further comprising:
determining an elapsed time in response to sending the first notification to the sensor; and
sending the second notification to the sensor based on the elapsed time.

7. The method of claim 2, further comprising:
receiving a user input for collecting continuous PPG data of the subject; and
sending the second notification to the sensor in response to receiving the user input.

8. The method of claim 1, further comprising:
determining whether the continuous PPG data is received in real-time from the sensor; and
sending the first notification to the sensor in response to determining that the continuos PPG data is not received in real-time.

9. A system comprising:
at least one processor;
a calculation module stored in a memory and executable by the at least one processor, the communication module configured to receive continuous photoplethysmographic (PPG) data of a subject from a sensor, calculate a continuous blood characteristic (BC) based on the continuous PPG data, and calculate a first quality metric of the continuous PPG data based on a sequence of the continuous BC; and
a determination module stored in the memory and executable by the at least one processor, the determination module communicatively coupled to the calculation module and configured to determine whether the first quality metric satisfies a stability criterion and send a first notification to the sensor in response to determining that the first quality metric satisfies the stability criterion, wherein the first notification instructs the sensor to collect compressed PPG data of the subject.

10. The system of claim 9, wherein the calculation module is further configured to receive compressed PPG data of the subject from the sensor in response to sending the first notification, calculate a compressed BC based on the compressed PPG data, and calculate a second quality metric of the compressed PPG data based on a sequence of the compressed BC.

11. The system of claim 10, wherein the determination module is further configured to determine whether the second quality metric satisfies the stability criterion and send a second notification to the sensor in response to determining that the second quality metric fails to satisfy the stability criterion, wherein the second notification instructs the sensor to collect continuous PPG data of the subject.

12. The system of claim 10, wherein the calculation module is further configured to calculate a standard deviation of the sequence of the compressed BC.

13. The system of claim 10, wherein the continuous BC and the compressed BC includes at least one of an oxygen saturation, a percentage modulation, and a heart rate.

14. The system of claim 10, wherein the calculation module is further configured to calculate an environmental signal from the compressed PPG data.

15. The system of claim 10, wherein the determination module is further configured to determine an elapsed time in response to sending the first notification to the sensor and send the second notification to the sensor based on an elapsed time.

16. A computer program product comprising a non-transitory computer readable medium encoding instructions that, in response to execution by at least one processor, cause the processor to perform operations comprising:
receive continuous photoplethysmographic (PPG) data of a subject from a sensor;
calculate a continuous blood characteristic (BC) based on the continuous PPG data;
calculate a first quality metric of the continuous PPG data based on a sequence of the continuous BC;
determine whether the first quality metric satisfies a stability criterion; and
send a first notification to the sensor in response to determining that the first quality metric satisfies the stability criterion, wherein the first notification instructs the sensor to collect compressed PPG data of the subject.

17. The computer program product of claim 16, further causing the processor to perform operations comprising:
receive compressed PPG data of the subject from the sensor in response to sending the first notification;
calculate a compressed BC based on the compressed PPG data;
calculate a second quality metric of the compressed PPG data based on a sequence of the compressed BC;
determine whether the second quality metric satisfies the stability criterion; and
send a second notification to the sensor in response to determining that the second quality metric fails to satisfy the stability criterion, wherein the second notification instructs the sensor to collect continuous PPG data of the subject.

18. The computer program product of claim 17, further causing the processor to calculate a standard deviation of the sequence of the compressed BC.

19. The computer program product of claim 17, further causing the processor to calculate an environmental signal from the compressed PPG data.

20. The computer program product of claim 17, further causing the processor to determine an elapsed time in response to sending the first notification to the sensor and send the second notification to the sensor based on the elapsed time.
